(19) United States
(12) Patent Application Publication
(10) Pub. No.: US 2017/0340351 A1
(43) Pub. Date: Nov. 30, 2017

(54) FLEXIBLE TROCAR ASSEMBLY FOR SURGICAL CIRCULAR STAPLING DEVICES

(71) Applicant: Covidien LP, Mansfield, MA (US)

(72) Inventor: Anthony Sgroi, JR., Wallingford, CT (US)

(21) Appl. No.: 15/466,900

(22) Filed: Mar. 23, 2017

Related U.S. Application Data
(60) Provisional application No. 62/343,193, filed on May 31, 2016.

(51) Int. Cl. A61B 17/34; A61B 17/072; A61B 17/115

(52) U.S. Cl. CPC A61B 17/3417; A61B 17/1155; A61B 17/07207; A61B 2017/07228; A61B 2017/07271; A61B 2017/07278; A61B 2017/07257

(57) ABSTRACT

An adapter assembly for connecting an end effector to a handle assembly includes a trocar assembly having a flexible trocar member.

### CROSS-REFERENCE TO RELATED APPLICATIONS

This application claims the benefit of and priority to U.S. Provisional Patent Application No. 62/343,193 filed May 31, 2016, the entire disclosure of which is incorporated by reference herein.

### BACKGROUND

### 1. Technical Field

The present disclosure relates generally to powered surgical circular stapling devices. More specifically, the present disclosure relates to flexible trocar members for powered surgical circular stapling devices.

### 2. Background of Related Art

Powered devices for use in surgical stapling procedures typically convert rotational motion from a handle assembly to linear motion for effectuating one or more functions, e.g., clamping, stapling, cutting. Powered surgical circular stapling devices generally include a trocar assembly having a rigid trocar member for supporting and approximating an anvil assembly relative to a cartridge assembly. During a surgical procedure, the anvil assembly is secured to a first section of tissue to be stapled and a second section of tissue is received over a cartridge assembly and is pierced by a rigid trocar member before the anvil assembly is secured to the rigid trocar member. The tight quarters of an operating room or the even tighter confines of a body cavity of a patient may increase the difficultly of securing the anvil assembly to the rigid trocar member.

Therefore, it would be beneficial to have a flexible trocar member for facilitating securing of an anvil assembly thereto.

### SUMMARY

Accordingly, a trocar assembly having a flexible portion is provided. The trocar assembly includes an outer housing defining a longitudinal axis, a trocar member supported within the outer housing and moveable along the longitudinal axis between a retracted position and an advanced position, and a drive screw rotatably supported relative to the outer housing and within the trocar member for moving the trocar member between the retracted and advanced positions. The trocar member includes a flexible portion.

In embodiments, the trocar member includes a trocar tip member and a base member, the flexible portion extends between and interconnects the trocar tip member and the base member. The flexible portion of the trocar member may include a flexible tubular member and a cable extending through the flexible tubular member. The flexible tubular member may include a coiled spring and the cable includes a flexible wire. The flexible portion may permit movement of the trocar tip member away from the longitudinal axis. The tubular member may permit transmission of distally directed forces to the trocar tip member. The cable may permit transmission of proximally directed forces to the trocar tip member. The flexible portion of the trocar member may be entirely received within the outer housing when in the retracted position. The flexible portion of the trocar member may be spaced from the outer housing when in the advanced position.

An adapter assembly for operably connecting a handle assembly to a tool assembly is also provided. The adapter assembly includes a connector portion configured for releasable connection to a handle assembly, an extension portion secured to the connector portion configured for releasable connection to a loading unit, and a trocar assembly supported within the extension portion configured for releasable connection to an anvil assembly. The trocar assembly includes a flexible trocar member.

In embodiments, the flexible trocar member includes a trocar tip member, a base member, and a flexible portion extending between and interconnecting the trocar tip member and the base member. The flexible portion of the trocar member may include a flexible tubular member and a cable extending through the flexible tubular member. The flexible tubular member may include a coiled spring and the cable includes a flexible wire.

The flexible portion may permit movement of the trocar tip member away from the longitudinal axis. The tubular member may permit transmission of distally directed forces to the trocar tip member. The cable may permit transmission of proximally directed forces to the trocar tip member. The flexible portion of the trocar member may be entirely received within the outer housing when in the retracted position. The flexible portion of the trocar member may be spaced from the outer housing when in the advanced position.

A surgical stapling device is also provided, and includes a handle assembly, an adapter assembly operably connectable to the handle assembly and including a flexible trocar member, and a tool assembly including an anvil assembly releasably connectable to the flexible trocar member.

### BRIEF DESCRIPTION OF THE DRAWINGS

Embodiments of the present disclosure are described herein with reference to the accompanying drawings, wherein:

FIG. 1 is a perspective view of an adapter assembly, in accordance with an embodiment of the present disclosure, an exemplary handle assembly, and an exemplary tool assembly;

FIG. 2 is a perspective side view of the adapter assembly shown in FIG. 1 including the exemplary tool assembly;

FIG. 3 is an enlarged cross-sectional view of the indicated area in FIG. 2, as taken through line 3-3 of FIG. 2;

FIG. 4 is an enlarged cross-sectional view of the indicated area in FIG. 2, as taken through line 4-4 of FIG. 2;

FIG. 5 is a perspective view of a trocar assembly of the adapter assembly shown in FIG. 1, with parts separated;

FIG. 6 is a perspective view of the trocar assembly shown in FIG. 5;

FIG. 7 is a cross-sectional side view taken along line 7-7 shown in FIG. 6, with a trocar member in a retracted position; and

FIG. 8 is a cross-sectional side view of the trocar assembly shown in FIG. 7, with the trocar member in an advanced position.

### DETAILED DESCRIPTION OF EMBODIMENTS

Embodiments of the presently disclosed adapter assembly for surgical stapling devices and/or handle assemblies are described in detail with reference to the drawings, in which like reference numerals designate identical or corresponding elements in each of the several views. As used herein the term “distal” refers to that portion of the adapter assembly or surgical stapling device, or component thereof, farther from the user, while the term “proximal” refers to that portion of the adapter assembly or surgical device, or component thereof, closer to the user.

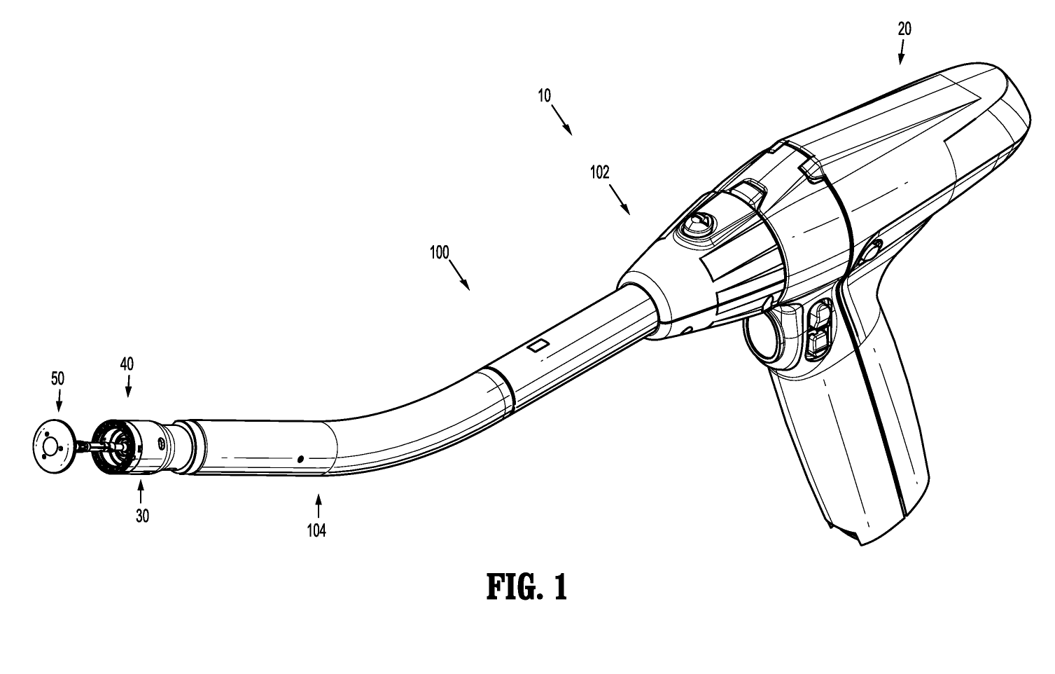

With reference to FIG. 1, an adapter assembly including a flexible trocar member in accordance with an embodiment of the present disclosure, shown generally as adapter assembly 100, is configured for selective connection to a powered handheld electromechanical instrument, shown generally as handle assembly 20. As illustrated in FIG. 1, the handle assembly 20 is configured for selective connection with the adapter assembly 100, and, in turn, the adapter assembly 100 is configured for selective connection with a tool assembly or end effector, e.g. tool assembly 30, which may, in exemplary embodiments, include a loading unit 40 and an anvil assembly 50, for applying a circular array of staples (not shown) to tissue (not shown). The handle assembly 20, along with the adapter assembly 100 and the tool assembly 30 form a surgical stapling device 10. Although the tool assembly 30 is shown and described for use with the adapter assembly 100, it is envisioned that the aspects of the present disclosure may be modified for use with stapling assemblies having alternative configurations.

For a detailed description of the structure and function of an exemplary handle assembly, please refer to commonly owned U.S. Pat. No. 9,055,943 (“the '943 patent”) and U.S. Pat. Appl. Publ. Nos. 2015/0157320 and 2015/0157321 (“the '320 and '321 applications”), the contents of which are incorporated herein by reference in its entirety.

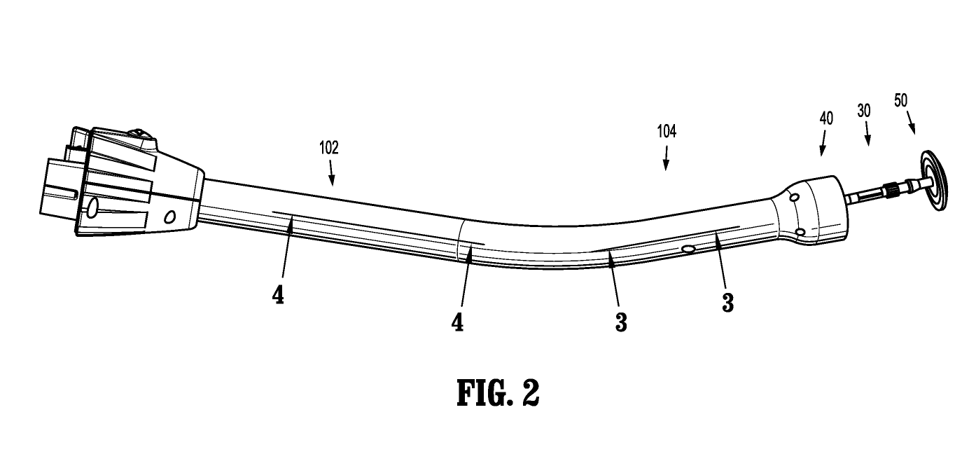

With additional reference to FIG. 2, the adapter assembly 100 includes a connection portion 102 configured for releasable connection to the handle assembly 20 (FIG. 1), and an extension portion 104 operably connected to the connection portion 102 for releasable connection to the loading unit 40 and the anvil assembly 50 of the tool assembly 30. The connection portion 102 and the extension portion 104 may be integrally formed, fixedly secured together, or releasably secured to one another. The adapter assembly 100 will only be described to the extent necessary to fully disclose the aspects of the present disclosure. For a detailed description of an exemplary adapter assembly including exemplary connector and extension portions, please refer to commonly owned U.S. patent application Ser. No. 14/875,766 (“the '766 application”), filed Oct. 21, 2014, the contents of which are incorporated herein by reference in their entirety, and the previously incorporated '943 patent and '320 and '321 applications.

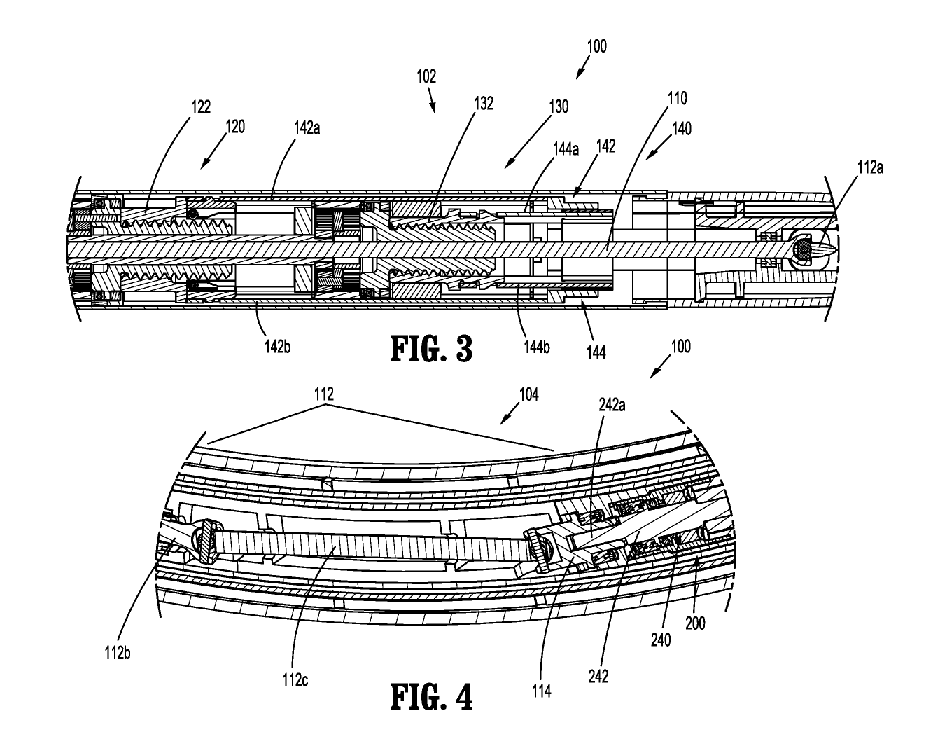

With additional reference to FIGS. 3 and 4, the adapter assembly 100 includes a drive shaft 110 extending through the connection portion 102. The drive shaft 110 includes a proximal end in operable connection with a first drive member (not shown) of the handle assembly 20 (FIG. 1) and a distal end in operable connection with a link assembly 112 (FIG. 4) supported within the extension portion 104 of the adapter assembly 100. The drive shaft 110 transfers rotational motion from the handle assembly 20 (FIG. 1) to the link assembly 112. As will be described in further detail below, the link assembly 112 is secured to a drive screw 240 of a flexible trocar assembly 200 and is configured such that rotation of the link assembly 112 causes longitudinal movement of a flexible trocar member 220 of the trocar assembly 200. Briefly, the link assembly 112 includes a plurality of link members 112a (FIG. 3), 112b (FIG. 4), 112c (FIG. 4), and a connector 114. For a detailed description of an exemplary link assembly, please refer to the '766 application.

With continued reference to FIG. 3, the connection portion 102 of the adapter assembly 100 includes a first pusher assembly 120 in operable connection with a second drive member (not shown) of the handle assembly 20 (FIG. 1) and a second pusher assembly 130 in operable connection with a third drive member (not shown) of the handle assembly 20. A first pusher member 122 of the first pusher assembly 120 is operably connected with first and second flexible bands 142a, 142b of an outer flexible band assembly 142 of a flexible band assembly 140. A second pusher member 132 of the second pusher assembly 130 is operably connected with first and second flexible bands 144a, 144b of an inner flexible band assembly 142 of the flexible band assembly 140. The first and second pusher assemblies 120, 130 operate to convert rotational motion from the respective first and second drive members (not shown) of the handle assembly 20 (FIG. 1) into longitudinal movement of the inner and outer flexible band assemblies 142, 144, respectively, of the flexible band assembly 140. In embodiments, the inner and outer flexible band assemblies 142, 144 of the flexible band assembly 140 are operably connected to a stapling assembly (not shown) and a knife assembly (not shown), respectively, within the loading unit 40 for stapling and cutting tissue, respectively.

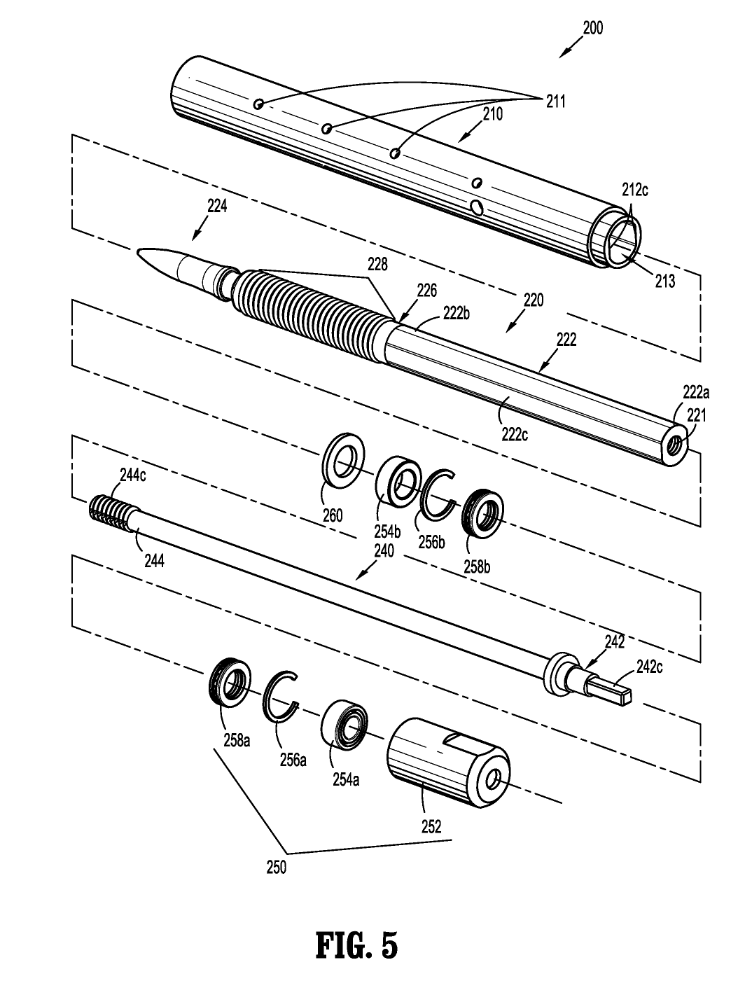

The trocar assembly 200 of the adapter assembly 100 will now be described with reference to FIGS. 5-8. The trocar assembly 200 includes an outer housing 210, a flexible trocar member 220 slidably disposed within the outer housing 210, and a drive screw 240 rotatably received within the trocar member 220 for longitudinally moving the trocar member 220 relative to the outer housing 210 along a longitudinal axis “x” of the trocar assembly 200. Although the flexible trocar member 220 will be shown and described as being a component of the trocar assembly 200, it is envisioned that the flexible trocar member 220 may be modified for use without a trocar assembly, and may instead be directly incorporated into circular staplers having alternative configurations.

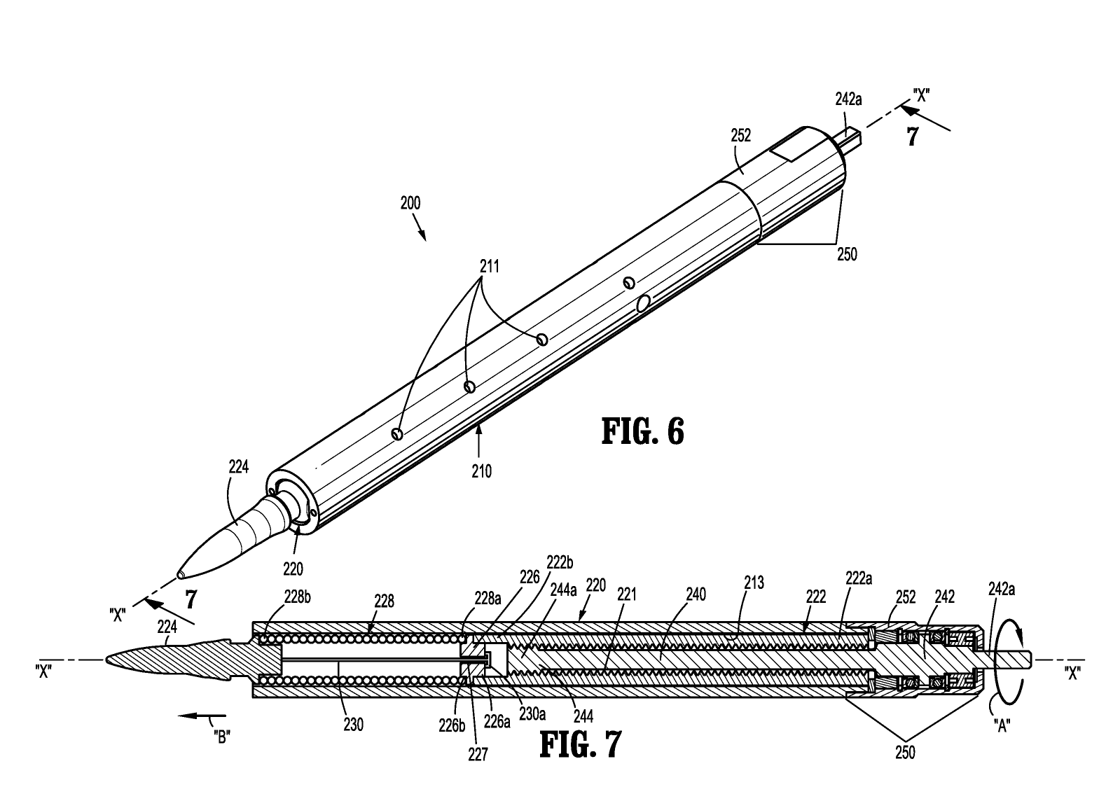

With particular reference to FIGS. 5 and 6, the outer housing 210 of the trocar assembly 200 is configured to be supported within the distal extension 104 (FIG. 2) of the adapter assembly 100 (FIG. 2). The outer housing 210 may include openings 211 for releasably or fixedly securing the outer housing 210 within the distal extension 104 of the adapter assembly 100. Alternatively, the outer housing 210 may include slots (not shown), tabs (not shown), or be otherwise configured to facilitate securing the outer housing 210 within the distal extension 104 of the adapter assembly 100.

The outer housing 210 of the flexible trocar assembly 200 defines a longitudinal opening or lumen 213 for slidably receiving the flexible trocar member 220. The outer housing 210 may include one or more flattened inner surfaces 212c (FIG. 5), tabs (not shown), notches (not shown) or be otherwise configured for engagement with a respective flattened outer surface 222c, slot (not shown), tabs (not shown) or other corresponding feature along at least a section of the length of the base member 222 of the flexible trocar member 220 to prevent the flexible trocar member 220 from rotating relative to the outer housing 210 as the drive screw 240 of the trocar assembly 200 is rotated within the flexible trocar member 220 to longitudinally translate the flexible trocar member 220.

With continued reference to FIGS. 5-7, the trocar member 220 of the adapter assembly 200 includes the base member 222 and a trocar tip member 224. A connector member 226 connects a flexible tubular member 228 with the base member 222, and the flexible tubular member 228 connects the trocar tip member 224 to the base member 222. A flexible cable 230 (FIG. 7) extends between and interconnects the connector member 226 and the trocar tip member 224. The flexible tubular member 228 and the flexible cable 230 form a flexible portion of the flexible trocar member 220.

The base member 222 of the flexible trocar member 220 of the trocar assembly 200 is slidably disposed within the outer housing 210 of the trocar assembly 200. As noted above, the base member 222 may include one or more flattened surfaces 222c corresponding to inner flattened surfaces 212c of the outer housing 210. The base member 222 defines a threaded longitudinal passage 221 for rotatably receiving the drive screw 240 of the trocar assembly 200. As will be described in further detail below, as the drive screw 240 rotates within the base member 222, the base member 222 moves longitudinally relative to the outer housing 210.

The connector member 226 of the flexible trocar member 220 of the trocar assembly 200 is secured to the distal end 222b of the base member 222 and operably connects the flexible tubular member 228 and the flexible cable 230 to the base member 222. As shown, the connector member 226 includes proximal and distal annular flanges 226a (FIG. 7), 226b (FIG. 7). The proximal flange 226a is received within a distal end of the threaded longitudinal passage 221 of the base member 222 and frictionally secures the connector member 226 with the base member 220. It is envisioned that the base member 222 and the connector member 226 may be releasably or permanently secured to one another in any suitable manner, including threading, slot and groove connection, adhering, welding. Alternatively, the connector member 226 may be integrally formed with the base member 222.

The distal flange 226b of the connector member 226 engages a proximal end 228a of the flexible tubular member 228. As shown, the connector member 226 defines an opening 227 through which a proximal end 230a of the flexible cable 230 is received. It is envisioned that the connector member 226 may include a hook, tab, or be configured in any other manner suitable for securing the proximal end 230a of the flexible cable 230 with the connector member 226.

As shown, the flexible tubular member 228 of the flexible trocar member 220 of the trocar assembly 200 includes a coil spring capable of bending with minimal force. It is envisioned that the flexible tubular member 228 may include semi-rigid tubing or other flexible tubular members capable of transferring an axial pushing force along the length thereof. A distal end 228b of the flexible tubular member 228 supports the trocar tip member 224. The trocar tip member 224 may be secured to the distal end 228b of the flexible tubular member 228 in any suitable manner.

The trocar tip member 224 of the flexible trocar member 220 of the trocar assembly 200 is configured for selective connection with an anvil assembly, e.g., the anvil assembly 50 (FIG. 1). As shown, the trocar tip member 224 is shorter than traditional trocar tips to accommodate the reduced space within a body cavity of a patient. It is envisioned, however, that the trocar tip member 224 may be modified to accommodate an anvil assembly of any size and/or any configuration.

The flexible cable 230 of the flexible trocar member 220 of trocar assembly 200 extends between and interconnects the connector member 226 and the trocar tip member 224. The flexible cable 230 may be secured to the trocar tip member 224 in any suitable manner. The flexible cable 230 may include a wire, string, cord or any flexible member capable of transferring an axial pulling force along the length thereof.

The drive screw 240 of the trocar assembly 200 is rotatably received within the flexible trocar member 220 of the trocar assembly 200. More particularly, a bearing assembly 250 is mounted to a proximal end 210a of the outer housing 210 of the trocar assembly 200 for rotatably supporting a proximal end 240a of drive screw 240 relative to outer housing 210 and flexible trocar member 220. The bearing assembly 250 includes a housing 252, proximal and distal spacers 254a, 254b, proximal and distal retention clips 256a, 256b, proximal and distal bearings 258a, 258b, and a washer 260.

As shown in FIG. 4, the proximal end 242 of the drive screw 240 includes a flange 242a configured for operable connection with the link assembly 112. A distal portion 244 of the drive screw 240 includes a threaded head 244a configured for threaded engagement with the threaded longitudinal passage 221 of the flexible trocar member 220. Although shown with only a section of the distal portion 244 of the drive screw 240 being threaded, it is envisioned that some or all of the length of the distal portion 244 of the drive screw 240 may be threaded.

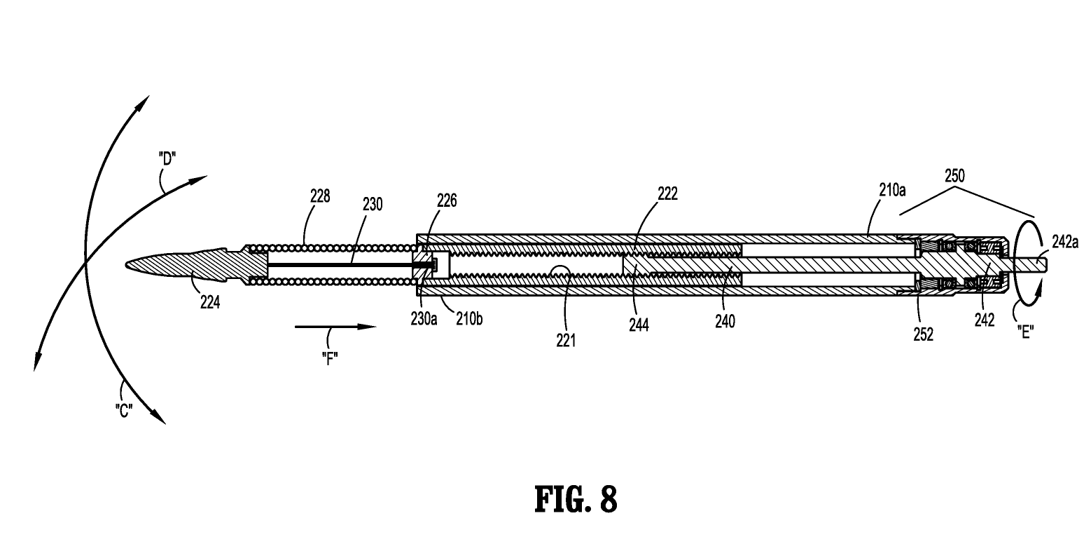

The operation of trocar assembly 200 will now be described with particular reference to FIGS. 7 and 8. Referring initially to FIG. 7, in an initial or retracted position, the flexible trocar member 220 of the trocar assembly 200 is received within the outer housing 210 of the trocar assembly 200 with the trocar tip member 224 of the flexible trocar member 220 extending from the outer housing 210. Although shown initially in the retracted position, it is envisioned that the trocar assembly 200 may be provided with the flexible trocar member 220 in an advanced position (FIG. 8).

As noted above, the trocar assembly 200 may be fixedly or releasably secured within the extension portion 104 (FIG. 1) of the adapter assembly 100 (FIG. 1). When the trocar assembly 200 is provided as a releasable unit, the trocar assembly 200 may be provided already secured within the adapter assembly 100 or may be provided separate from the adapter assembly 100, thereby requiring attachment of the trocar assembly 200 to the adapter assembly 100.

Once the trocar assembly 200 is secured to the adapter assembly 100, the surgical stapling device 10 operates in a traditional manner. During use, the flexible trocar member 220 of the trocar assembly 200 is extended by actuating the handle assembly 20 (FIG. 1) to cause rotation of the first drive member thereof (not shown) which causes rotation of the drive shaft 110 of the adapter assembly 100. As the drive shaft 110 rotates, the link assembly 112 transfers the rotational force from the drive shaft 110 to the drive screw 240 of the trocar assembly 200. As the drive screw 240 rotates in a first direction, as indicated by arrow “A” in FIG. 7, the flexible trocar member 220 of the trocar assembly 200 moves in a distal direction, e.g., advances, as indicated by arrow “B” in FIG. 7. The semi-rigid nature of the flexible tubular member 228 of the flexible trocar member 220 enables the pushing action of the flexible trocar member 220 during advancement of the flexible trocar member 220.

Turning now to FIG. 8, the flexible trocar member 220 is shown in the advanced or extended position. When in the advanced position, the flexible nature of the flexible tubular member 226 and the flexible cable 230 of the flexible trocar member 220, e.g., the flexible portion of the flexible trocar member 220, enables the trocar tip member 224 of the flexible trocar member 220 to move in a side-to-side direction, as indicated by arrow “C”, in a front-to-back direction, as indicated by arrow “D”, and in any direction (e.g., omni-directionally) therebetween. The ability to move the trocar tip member 224 in the various directions facilitates attachment of the anvil assembly 50 (FIG. 2) to the trocar tip member 224.

Once the anvil assembly 50 (FIG. 2) is secured to the trocar tip member 224 of the trocar assembly 200, the handle assembly 20 (FIG. 1) is actuated to cause the first drive member (not shown) to rotate in a second direction, as indicated by arrow “E” in FIG. 8, to move the flexible trocar member 220 in the proximal direction, e.g., retract, as indicated by arrow “F” in FIG. 8. The flexible cable 230 of the flexible trocar member 220 enables the pulling action of the flexible trocar member 220 during retraction of the flexible trocar member 220.

After the surgical stapling device 10 has been fired and the tissue to be stapled has been stapled and cut, the anvil assembly 50 is moved away from the loading unit 40 through operation of the handle assembly 20 (FIG. 1) and the trocar assembly 200, as described above. The flexible nature of the flexible portion of the flexible trocar member 220 may also facilitate removal of the anvil assembly 50 from the flexible trocar member 220.

It is envisioned that the trocar assembly 200 may be reusable. Thus, following the stapling procedure, the trocar assembly 200 may be removed from the extension portion 104 of the adapter assembly 100 and sterilized. Alternatively, the trocar assembly 200 may be disposed of in a customary manner.

Any of the components described herein may be fabricated from either metals, plastics, resins, composites or the like taking into consideration strength, durability, wearability, weight, resistance to corrosion, ease of manufacturing, cost of manufacturing, and the like.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A trocar assembly for a surgical stapling device, the trocar assembly comprising:
an outer housing defining a longitudinal axis;
a trocar member supported within the outer housing and moveable along the longitudinal axis between a retracted position and an advanced position, wherein the trocar member includes a flexible portion; and
a drive screw rotatably supported relative to the outer housing and within the trocar member for moving the trocar member between the retracted and advanced positions.

2. The trocar assembly of claim 1, wherein the trocar member includes a trocar tip member and a base member, the flexible portion extends between and interconnects the trocar tip member and the base member.

3. The trocar assembly of claim 1, wherein the flexible portion of the trocar member includes a flexible tubular member and a cable extending through the flexible tubular member.

4. The trocar assembly of claim 3, wherein the flexible tubular member includes a coiled spring and the cable includes a flexible wire.

5. The trocar assembly of claim 1, wherein the flexible portion permits movement of the trocar tip member away from the longitudinal axis.

6. The trocar assembly of claim 3, wherein the tubular member permits transmission of distally directed forces to the trocar tip member.

7. The trocar assembly of claim 3, wherein the cable permits transmission of proximally directed forces to the trocar tip member.

8. The trocar assembly of claim 3, wherein the flexible portion of the trocar member is entirely received within the outer housing when in the retracted position.

9. The trocar assembly of claim 3, wherein the flexible portion of the trocar member is spaced from the outer housing when in the advanced position.

10. An adapter assembly for operably connecting a handle assembly to a tool assembly, the adapter assembly comprising:
a connector portion configured for releasable connection to a handle assembly;
an extension portion secured to the connector portion configured for releasable connection to a loading unit; and
a trocar assembly supported within the extension portion configured for releasable connection to an anvil assembly, wherein the trocar assembly includes a flexible trocar member.

11. The adapter assembly of claim 10, wherein the flexible trocar member includes a trocar tip member, a base member, and a flexible portion extending between and interconnecting the trocar tip member and the base member.

12. The adapter assembly of claim 10, wherein the flexible portion of the trocar member includes a flexible tubular member and a cable extending through the flexible tubular member.

13. The adapter assembly of claim 12, wherein the flexible tubular member includes a coiled spring and the cable includes a flexible wire.

14. The adapter assembly of claim 10, wherein the flexible portion permits movement of the trocar tip member away from the longitudinal axis.

15. The adapter assembly of claim 12, wherein the tubular member permits transmission of distally directed forces to the trocar tip member.

16. The adapter assembly of claim 12, wherein the cable permits transmission of proximally directed forces to the trocar tip member.

17. The adapter assembly of claim 12, wherein the flexible portion of the trocar member is entirely received within the outer housing when in the retracted position.

18. The adapter assembly of claim 12, wherein the flexible portion of the trocar member is spaced from the outer housing when in the advanced position.

19. A surgical stapling device comprising:
a handle assembly;
an adapter assembly operably connectable to the handle assembly and including a flexible trocar member; and
a tool assembly including an anvil assembly releasably connectable to the flexible trocar member.
